(19) United States
(12) Patent Application Publication
(10) Pub. No.: US 2024/0311638 A1
(43) Pub. Date: Sep. 19, 2024

(54) NATURAL KILLER CELL EFFICACY PREDICTION METHOD AND COMPUTING DEVICE

(71) Applicant: Industrial Technology Research Institute, Hsinchu (TW)

(72) Inventors: Nien-Tzu Chou, Hsinchu City (TW); Yu-Yu Lin, Hsinchu County (TW); Ching-Fang Lu, Hsinchu County (TW); Jian-Hao Li, Hsinchu City (TW); Ting-Hsuan Chen, Hsinchu County (TW); Cheng-Tai Chen, Taoyuan City (TW)

(73) Assignee: Industrial Technology Research Institute, Hsinchu (TW)

(21) Appl. No.: 18/398,161

(22) Filed: Dec. 28, 2023

Related U.S. Application Data
(60) Provisional application No. 63/435,556, filed on Dec. 28, 2022.

(51) Int. Cl. G06N 3/0895; G01N 33/50; G16B 40/20

(52) U.S. Cl. CPC G06N 3/0895; G01N 33/5023; G16B 40/20; G01N 2333/70503; G01N 2333/70596; G01N 2333/7155

(57) ABSTRACT

A method of predicting the efficacy of natural killer cells, including: generating a plurality of training data corresponding to a plurality of donors based on a characteristic factor and a corresponding killing result against the target cancer cells of a plurality of cultured natural killer cells from the donors; obtaining a trained neural network model by inputting the plurality of training data into a neural network model; inputting a to-be-tested input vector corresponding to at least one characteristic factor of a to-be-tested natural killer cell into the trained neural network model to obtain an outputted result vector of the trained neural network model, wherein the result vector indicates a predicted killing result corresponding to the target cancer cell after applying the to-be-tested natural killer cell; and determining a quality of the to-be-tested natural killer cell based on the predicted killing result.

### CROSS-REFERENCE TO RELATED APPLICATION

This application claims the priority benefits of U.S. provisional application Ser. No. 63/435,556, filed on Dec. 28, 2022. The entirety of the above-mentioned patent applications is hereby incorporated by reference herein and made a part of this specification.

### BACKGROUND

### Technical Field

The present disclosure relates to a prediction method and to a method and computing device for predicting the performance of natural killer cell.

### Description of Related Art

Among the many strategies against cancer, immunocellular therapy is particularly notable, especially natural killer cell (NK) therapy. Because of their inherent advantages of not requiring antigen recognition and not posing a risk of graft-versus-host disease (GVHD), they are considered to have great potential for the development of allogeneic cell-based therapeutic products. However, the development of NK cell therapies is challenging because of the significant variation in the function of NK cells between individuals and the limited number of NK cells in the bloodstream. This requires in vitro culture and expansion until sufficient numbers are available, as well as a series of functional analyses to confirm the efficacy of the NK cells. The time-consuming nature of the process and the high cost of materials are factors that limit the further development of the cellular therapy industry.

### SUMMARY

Based on the foregoing background and motivation, the present disclosure provides a method for predicting function of natural killer cells cultured in vitro. The comprehensive information obtained by analyzing the basic properties of NK cell products can be used to predict the efficacy of NK cells, which can also be used as a condition for quality control of cell products, so as to improve production efficiency and quality.

A method for predicting the performance of a Natural Killer (NK, Natural Killer) cell, comprising: generating a plurality of training data according to a characteristic factor and a corresponding killing result against a target cancer cell of each of the natural killer cells; obtaining a trained neural network model by inputting the plurality of training data into a neural network model; inputting a to-be-tested input vector corresponding to at least one characteristic factor of a to-be-tested natural killer cell into the trained neural network model to obtain a result vector outputted by the trained neural network model, wherein the result vector indicates a predicted killing result corresponding to the target cancer cell after applying the to-be-tested natural killer cell; and determining a quality of the to-be-tested natural killer cell according to the predicted killing result.

In an embodiment of the present disclosure, wherein the characteristic factor comprises: a plurality of killer cell activating receptor (KAR) characteristic values, where the KAR characteristic values are expression ratios of the KARs, wherein the killing result comprises: a reduction proportion of the target cancer cell.

In an embodiment of the present disclosure, wherein the step of obtaining the trained neural network model by inputting the plurality of training data into the neural network model comprises: generating a training first vector corresponding to each natural killer cell according to a characteristic factor of each natural killer cell in the plurality of training data; generating a training second vector corresponding to the training first vector according to the killing result of each natural killer cell in the plurality of training data; and inputting the training first vector and the training second vector into the neural network model to obtain the trained neural network model through a supervised learning algorithm.

In an embodiment of the present disclosure, wherein the supervised learning algorithm comprises one of the following: a multilayer perceptron (MLP), consisting of a plurality of layers, each layer has a plurality of nodes, wherein the last layer has only one node and an output of the node of the last layer is the result vector; and a deep learning networks, comprising convolutional neural networks and recurrent neural networks (RNN).

In an embodiment of the present disclosure, wherein the target cancer cell comprises one of the following types of cancer cells: a triple-negative breast cancer cell (MDA-MB-231) and a leukemia cancer cell (K562).

In an embodiment of the present disclosure, wherein when the target cancer cell is the triple-negative breast cancer cell (MDA-MB-231), the plurality of KAR characteristic values comprises characteristic values corresponding to at least one of the following KARs: NKG2D, CD226, and CD25, wherein the NKG2D, the CD226, and the CD25 are listed based on associated weights in descending order, wherein when that target cancer cell is the leukemia cancer cell (K562), the plurality of KAR characteristic values comprises characteristic values corresponding to at least one of the following KARs: CD226, NKp46, and CD16, wherein the CD226, the NKp46, and the CD16 are listed based on the associated weights in descending order.

In an embodiment of the present disclosure, wherein when the target cancer cell is the triple-negative breast cancer cell (MDA-MB-231), the plurality of KAR characteristic values further comprises characteristic values corresponding to at least one of the following KARs: CD16, CD56, CD69, NKp30, NKp44, and NKp46. When that target cancer cell is the leukemia cancer cell (K562), the plurality of KAR characteristic values further comprises characteristic values corresponding to at least one of the following KARs: CD25, CD56, CD69, NKp30, NKp44, and NKG2D.

In an embodiment of the present disclosure, wherein the plurality of KAR characteristic values further comprises characteristic values corresponding to at least one of the following KARs: 2B4, NKG2A, NKG2C, CD158a/b, CD57, CD62L, CD161, NKp80 and 4-1BB.

In an embodiment of the present disclosure, wherein the step of determining quality of the to-be-tested natural killer cell according to the predicted killing result comprises: when a predicted reduction proportion of the target cancer cell is greater than or equal to A, the quality of the to-be-tested natural killer cell is determined to be good; when a predicted reduction proportion of the target cancer cell is less than A and greater than B, the quality of the to-be-tested natural killer cell is determined to be medium; and when a predicted reduction proportion of the target cancer cell is less than or equal to B, the quality of the to-be-tested natural killer cell is determined to be poor, wherein A is greater than B.

In an embodiment of the present disclosure, wherein A and B are predetermined according to the following variables: the KAR characteristic values and types of target cancer cells, wherein the KAR characteristic values comprise: expression ratios of markers of KARs of a natural killer cell.

In an embodiment of the present disclosure, wherein when the target cancer cell is triple-negative breast cancer cells (MDA-MB-231), A is 70% and B is 40%, when the target cancer cell is leukemia cancer cells (K562), A is 50% and B is 36%.

A further embodiment of the present disclosure provides a computing device, adapted for predicting performance of a natural killer (NK) cell. The computing device comprises: a processor, wherein the processor is configured to: generate a plurality of training data according to a characteristic factor and a corresponding killing result against a target cancer cell of each of the natural killer cells; obtain a trained neural network model by inputting the plurality of training data into a neural network model; input a to-be-tested input vector corresponding to at least one characteristic factor of a to-be-tested natural killer cell into the trained neural network model to obtain a result vector outputted by the trained neural network model, wherein the result vector indicates a predicted killing result corresponding to the target cancer cell after applying the to-be-tested natural killer cell; and determine a quality of the to-be-tested natural killer cell according to the predicted killing result.

Based on the above, the present disclosure provides an innovative predictive method and computing device designed to evaluate the killing ability of natural killer cells, provided by different donors, against cancer cells. The method starts by collecting natural killer cell samples from multiple donors and identifying their killing results on the target cancer cells as well as the characteristic factors they possess. These data are used as training data and fed into a neural network model that is trained to learn and recognize the relationship between different characteristic factors and the effects of poisoning. After the model has been trained, the characteristic factors of any to-be-tested natural killer cells can be input into the model. The model then outputs a result vector that provides a predicted killing performance, which allows the researcher or physician to evaluate the potential therapeutic performance of the to-be-tested natural killer cells against specific cancer cells. This prediction not only enhances the potential for individualization of clinical treatments, but also accelerates the screening process for effective treatment options. In addition, after obtaining the predicted quality of each of the to-be-tested natural killer cells, the best or better quality of one or more of the to-be-tested natural killer cells can be selected for the actual application of the natural killer cell immunotherapy program to cancer patients, such that the outcome and quality of life of cancer patients will be greatly improved.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a block schematic diagram of a computing device, depicted according to an embodiment of the present disclosure.

FIG. 2A is a schematic diagram of data stored in a storage circuit unit depicted according to an embodiment of the present disclosure.

FIG. 2B is a schematic diagram of data stored in a database, depicted according to an embodiment of the present disclosure.

FIG. 3 is a flowchart of a method for predicting the performance of natural killer cells, depicted according to an embodiment of the present disclosure.

FIG. 4 is a schematic diagram of a method for predicting the performance of natural killer cells, depicted according to an embodiment of the present disclosure.

FIG. 5A is a schematic diagram of comparing actual results with predicted results for K562 cancer cell, depicted according to an embodiment of the present disclosure.

FIG. 5B is a schematic diagram comparing actual results with predicted results for MDA_MB_231 cancer cell, depicted according to an example of an embodiment of the present disclosure.

### DESCRIPTION OF THE EMBODIMENTS

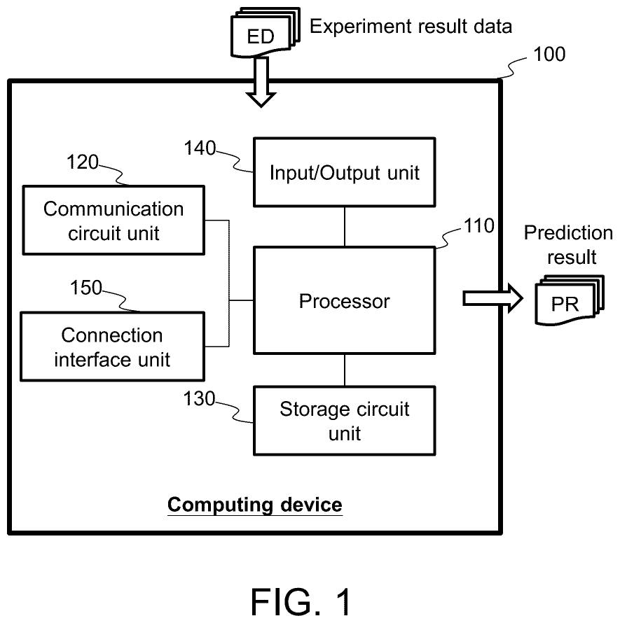

Referring to FIG. 1, in the embodiment, the computing device 100 includes a processor 110, a communication circuit unit 120, a storage circuit unit 130, an input/output unit 140, and a connection interface unit 150.

The processor 110 is, for example, a Microprogrammed Control Unit (MCU), a Central Processing Unit (CPU), a programmable microprocessor, an Application Specific Integrated Circuits (ASIC), a Programmable Logic Device (PLD), or other similar device.

The communication circuit unit 120 is coupled (electrically connected) to the processor 110 to establish a network connection via wireless communication to connect to the Internet or other electronic devices, so as to transmit or receive data. In the embodiment, the communication circuit unit 120 may have a wireless communication circuit module (not shown) and support one or a combination of Global System for Mobile Communication (GSM) systems, Wireless Fidelity (WiFi) systems, different generations of mobile communication technologies (e.g., 3G˜6G), Bluetooth communication technologies, but it is not limited thereto. In another embodiment, the communication circuit unit 120 further includes a wired communication circuit module to establish a network connection, e.g., to an Internet or other electronic device, by means of a wire.

The storage circuit unit 130 is coupled to the processor 110. The storage circuit unit 130 may store data as directed by the processor 110. The storage circuit unit 130 includes a hard disk drive (HDD) or a non-volatile memory storage device (e.g., SSD) of any type. In one embodiment, the storage circuit unit 130 further includes memory, such as Dynamic Random Access Memory (DRAM), Static Random Access Memory (SRAM), and the like, for temporarily storing instructions or data executed by the processor.

The experiment result data ED may be received via the communication circuit unit 120 or the connection interface unit 150, or be inputted via the input/output unit 140. The generated prediction results PR may be transmitted to other electronic devices via the communication circuit unit 120 or the connection interface unit 150, or be displayed via the input/output unit 140. In addition, both the experiment result data ED and the prediction result PR may be stored in a database in the storage circuit unit 130.

The input/output unit 140, coupled to the processor 110, includes an input device and an output device. Input devices, such as microphones, touchpads, touch panels, keyboards, mouse, etc., are used to allow the user to enter data or to control the functions desired by the user. Output devices include, for example, monitors, speakers, etc., but it is not limited thereto. In an embodiment, the input/output unit 140 may be a touch screen, a head-up display, or a head-mounted display. The results/data generated by the computing device 100 may be displayed on the display.

The connection interface unit 150 is coupled to the processor 110, the processor 110 is configured to establish data connections with other electronic devices or storage devices via the connection interface unit 150 to transmit data.

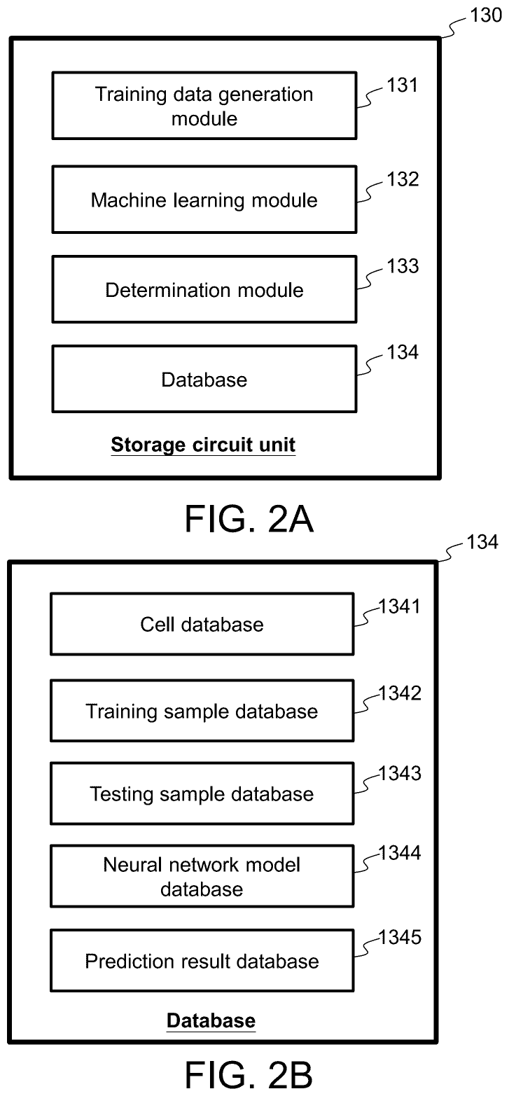

Referring to FIG. 2A, the data stored in the storage circuit unit 130 includes a plurality of program/code modules and databases. The program/code modules are, for example, a training data generation module 131, a machine learning module 132, and a determination module 133.

The training data generation module 131 is configured to process any procedure regarding the generation of training data according to experiment result data. The training data generation module 131 can remove unnecessary content from the experiment result data, leaving the desired content (also known as target content). Experiment result data is, for example, data recording the results of experiments on natural killer cells (hereinafter referred to as NK cells) of donors. Values from different batches of the same donor are considered as independent data. Target contents are, for example, characteristic factors, killing ability and cell function of a NK cell. The characteristic factors are, for example, cell purity, cell viability, characteristic values of Killer cell Activating Receptor of different NK cell (KAR characteristic values), amplification factor, and the ability to kill target cancer cells. The NK cell KAR characteristic value is, for example, an expression ratio (%) of a KAR marker (or a KAR marker protein expression ratio). In this embodiment, the amount of target protein expressed on each natural killer cell is sequentially analyzed by using a flow cytometer through the channel. For example, Gate circles 10,000 natural killer cells (as 100%), of which 3,000 natural killer cells have target KAR on their surface (30%). In this example, the expression ratio for the target KAR is 30%. Another example is that the expression ratio can be measured by means of the MFI average fluorescence intensity: multiple natural killer cells are passed through the measurement channel to detect the relative intensity fluorescence value of the corresponding target KAR of each natural killer cell, e.g., the relative intensity fluorescence values of multiple natural killer cells were collected to calculate the corresponding average fluorescence intensity as the KAR expression ratio of these cells.

In this embodiment, the killing ability (also referred to as the killing result) comprises: a reduction proportion (%) of the target cancer cells after application of the NK cells to the target cancer cell. In an embodiment, the cell function further includes one or more of the following: an expression ratio (%) of IFN-γ after co-culture with the target cancer cell, an expression ratio (%) of TNF-α after co-culture with the target cancer cell, and an expression ratio (%) of CD107a after co-culture with the target cancer cell.

In evaluating the functionality of NK cells, consideration is given not only to their direct killing ability against the target cancer cells, but also to the expression levels of immunomodulatory factors released under a condition that the NK cells co-culture with the target cancer cell. Specifically, the expression ratios (%) of the aforementioned immunomodulatory factors include the expression ratios (%) of interferon-gamma (IFN-γ) and tumor necrosis factor-alpha (TNF-α) and the expression ratio of the cell-surface degranulation marker CD107a produced by NK cells after co-culture with the target cancer cells. These indicators are beneficial to reflect the immune response ability of NK cells to the target cancer cell and their potential therapeutic effects.

CD107a, as a cell surface molecule, is an important degranulation particle indicator marker for NK cells undergoing effector activation. When NK cells attack against their target cell, i.e., tumor or infected cells, their particles containing toxic molecules move to the cell membrane of the NK cells and fuse with the cell membrane. During this process, CD107a is translocated to the surface of the NK cell membrane, which is a signal for the release of granule contents, such as a variety of cytotoxic proteins, to the outside of the cell. The release of these proteins ultimately leads to the death of the target cell. Therefore, the expression of CD107a can be used as an experimental marker to evaluate the cytotoxic activity of NK cells.

The expression ratio of CD107a as an indicator of NK cell cytotoxicity release may be tested by flow cytometric analysis. In this experiment, effector NK cells cultured for 14 days were co-cultured with the target tumor cells K562 and MDA-MB-231 at a ratio of 1:1 for a four hours killing experiment. Four hours after the end of the experiment, simultaneous double staining for CD56 and CD107a was performed to assess the cytotoxic function of NK cells. This analysis was performed to determine the ability of NK cells to release cytotoxins upon contact with the target tumor cells.

In an embodiment, the generated training data includes characteristic factors and the killing results corresponding to different NK cells.

In one embodiment, the target cancer cells comprise one of the following plurality of cancer cells: triple negative breast cancer cells (MDA-MB-231) and leukemia cancer cells (K562).

In this embodiment, high quality natural killer cells were successfully grown in a 14-day precision culture process via human peripheral blood mononuclear cells (PBMC) extracted from healthy human blood samples and using ITRI-NK magnetic beads in conjunction with a series of selected cytokines. These NK cells not only demonstrated high viability and purity of over 90%, but also achieved an amplification factor (expansion rate) of over 1000 times. After two weeks of activation, these NK cells exhibited significant KAR activity and good killing ability, and were able to secrete large quantities of the immunoregulatory factors IFN-γ and TNF-α, which are indispensable properties for their anti-tumor response.

The procedure for detection of KAR expression ratio includes: immunostaining performed on NK cells cultured for 14 days using a variety of antibody markers, including CD56, CD158a/b, CD16, CD69, CD25, NKG2D, CD226, NKp46, NKp44, NKp30, 2B4, NKG2C and NKG2A. Next, flow cytometry was utilized to quantify the degree of expression of these NK cell surface factors, such as CD56+ expression along with the percentage of expression of each of the other markers. This analysis facilitates understanding of the phenotypic characteristics and functional potential of NK cells after activation.

NK cells, as the body's defense mechanism, have different “weapons” against cancer cells. These weapons can be categorized into three types:

(1) Activating receptor (+): This type is an activation marker on NK cells that promote the anti-tumor activity of NK cells. When the “switches” on these NK cells are turned on, they can attack and stop tumor cells from growing. These “switches” include NKG2D (Nature Killer Group 2D), NKp46 (also known as CD335, Cluster of Differentiation 335), NKp44 (also known as CD336, Cluster of Differentiation 336), NKp30 (also known as CD337, Cluster of Differentiation 337), CD16 (Cluster of Differentiation 16) and CD161 (Cluster of Differentiation 161), etc.

(2) Co-activating receptor (+): This type is a co-stimulatory molecule on the surface of NK cells, which further enhances the ability to act as a co-stimulator against tumors. It can be regarded as a “booster” to enhance the attack power of NK cells. They help NK cells fight tumors better, and these co-stimulatory molecules include NKp80 (killer cell lectin-like receptor subfamily F, member 1 (KLRF1)), DNAM-1 (CD226, Cluster of Differentiation 226), and 2B4 (CD244, Cluster of Differentiation 244).

(3) Inhibitory receptor (−): This type of inhibitory marker on NK cells may promote tumor growth, and these receptors are key factors in the mechanism by which tumors escape immune surveillance. These receptors act as “brakes” for NK cells, slowing or stopping them from attacking, and sometimes even helping tumor cells escape. These “brakes” include NKG2A/CD94 (a type of natural killer cell receptor), and KIRs (CD158a/b, Cluster of Differentiation 158a/b).

The ways to improve the anti-cancer ability of NK cells include enhancing the activity of the KAR (activating receptor) on the NK cells to allow the NK cells to attack the cancer cells more aggressively and reducing the activity of KIR (inhibitory receptor). In this way, NK cells will then not be tricked by the cancer cells into slowing down their attack.

In the immune response of natural killer cells (NK cells), expression of CD25 (Cluster of Differentiation 25) and CD69 (Cluster of Differentiation 69) molecules is a key marker for distinguishing the stage of NK cell activation. CD25 is commonly associated with the early activation of NK cells, which are at a stage with the potential to develop into memory-type NK cells. These memory-type NK cells have characteristics similar to those of memory T cells in the adaptive immune response and can remember previously encountered antigens, resulting in producing a faster and more effective response when encountering again. Compared with CD25, CD69 is associated with the late activation of NK cells, which show higher cytotoxic activity and readiness to fight against tumors or infections. The dynamic changes of these molecules provide insights into the functional state of NK cells and the process of immune regulation.

The machine learning module 132 is configured to perform machine learning operations according to the generated training data (also known as training samples). The determination module 133 is configured to determine the quality or efficacy of NK cells according to the predictions outputted by the neural network model.

Referring to FIG. 2B, the database 134 is, for example, a cell database 1341, a training sample database 1342, a test sample database 1343, a neural network model database 1344, and a prediction results database 1345.

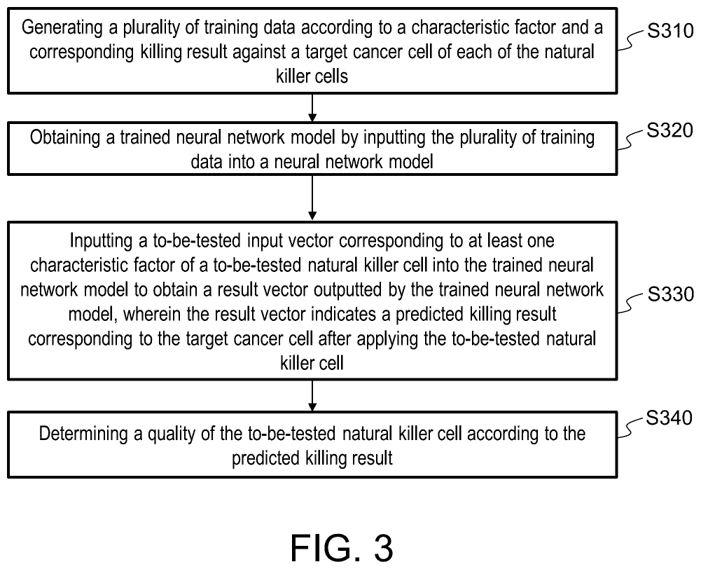

Referring to FIG. 3, in step S310, the processor 110 generates a plurality of training data according to a characteristic factor and a corresponding killing result against a target cancer cell of each of the natural killer cells. In one embodiment, the plurality of natural killer cells correspond to multiple donors respectively.

In this embodiment, for illustrative purposes, the characteristic factor is the KAR characteristic value. The KAR characteristic value can be considered as the expression ratio (%) of the KAR marker.

When the target cancer cell is triple-negative breast cancer cell (MDA-MB-231), the plurality of KAR characteristic values include characteristic values corresponding to at least one of the following KARs: NKG2D, CD226, and CD25, wherein the NKG2D, the CD226, and the CD25 are listed based on the associated weighting in descending order. That is, for MDA-MB-231, the expression ratio of the NKG2D with the highest importance weight has the greatest influence on the reduction proportion of MDA-MB-231 (i.e., NK cells with higher expression ratios of NKG2D have higher chances to obtain a higher reduction proportion of MDA-MB-231). Further, when the target cancer cell is leukemia cancer cell (K562), the plurality of KAR characteristic values include characteristic values corresponding to at least one of the following KARs: CD226, NKp46, and CD16, wherein the CD226, the NKp46, and the CD16 are listed based on the associated weights in a descending order.

For example, assume that the target cancer cell is leukemia cancer cell (K562). After applying a type of NK cell to co-culture with K562, the percentage of K562 cells decreased from 100% to 30%, indicating that the killing ability of this type of NK cell was 70% (i.e., the reduction proportion of the target cancer cells was 100%−30%=70%).

In an embodiment, when the target cancer cell is the triple-negative breast cancer cell (MDA-MB-231), the plurality of KAR characteristic values further include characteristic values corresponding to at least one of the following KARs: CD16, CD56, CD69, NKp30, NKp44 and NKp46. In addition, when the target cancer cell is the leukemia cancer cell (K562), the plurality of KAR characteristic values further include characteristic values corresponding to at least one of the following KARs: CD25, CD56, CD69, NKp30, NKp44 and NKG2D.

Further, in another embodiment, the plurality of KAR characteristic values further include characteristic values corresponding to at least one of the following KARs: 2B4, NKG2A, NKG2C, CD158a/b, CD57, CD62L, CD161, NKp80 and 4-1BB.

In step S320, the processor 110 obtains a trained neural network model by inputting the plurality of training data into a neural network model.

In step S330, the processor 110 inputs a to-be-tested input vector corresponding to at least one characteristic factor of a to-be-tested natural killer cell into the trained neural network model to obtain a result vector outputted by the trained neural network model, in which the result vector indicates a predicted killing result corresponding to the target cancer cell after applying the to-be-tested natural killer cell.

In this embodiment, the machine learning module 132 uses a manner of supervised learning by inputting a plurality of training data into the neural network model for training to obtain a trained neural network model. More specifically, a training first vector corresponding to each natural killer cell can be generated according to the characteristic factor of each natural killer cell in the plurality of training data. Next, a training second vector corresponding to the training first vector can be generated according to the killing result of each natural killer cell in the plurality of training data, and the training the first vector and the training the second vector can be inputted into the neural network model through supervised learning algorithm to obtain the trained neural network model.

The supervised learning algorithm comprises one of the following: a multilayer perceptron (MLP), consisting of a plurality of layers, each layer has a plurality of nodes, in which the last layer has only one node and an output of the node of the last layer is the result vector; and

a deep learning networks, comprising convolutional neural networks (CNN) and recurrent neural networks (RNN).

For example, the neural network model used has an input layer, a hidden layer, and an output layer. The input layer has P nodes, the hidden layer has Q nodes, and the output layer has 1 node. The number of P's is equal to the number of types of KAR characteristic values of NK cell. For example, if the experiment result data records 9 KAR characteristic values for each of different NK cells, P could be set to 9. The 9 KAR characteristic values may be input to the input layer as an input vector (e.g., the 9 KAR characteristic values may be assembled as a training first vector or a to-be-tested input vector), in which each node is input with one of the KAR characteristic values of the input vector. Q is, for example, 1, 2, 3 or 4, depending on different algorithms. The output layer is used to output the predicted results, e.g., the predicted reduction proportion (%) of the target cancer cells. The higher the reduction proportion means the better the predicted result (predicted killing result) outputted. In another embodiment, the hidden layer may have two layers, the first layer may have 1 to 4 nodes and the second layer may have 0 to 4 nodes. The root-mean-square error (RMSE) of the prediction results for different combinations of hidden layers and number of nodes can be compared using bootstrap statistics, and the combination with the lowest RMSE can be selected as the modeling architecture to be used in practice. In one embodiment, the combination with the lowest root mean square error is that the first layer of the hidden layer has 4 nodes and the second layer has 0 nodes (i.e., only the first layer). In another embodiment, the combination with the second lowest root mean square error is that the first of the hidden layers has 4 nodes and the second layer has 4 nodes.

It should be noted that, in an embodiment, the processor 110 may generate retraining data according to the obtained predicted killing result for the to-be-tested NK cells and the plurality of KAR characteristic values for the to-be-tested NK cells. The retraining data is also utilized to retrain the trained neural network model in order to enhance the accuracy of the trained neural network model.

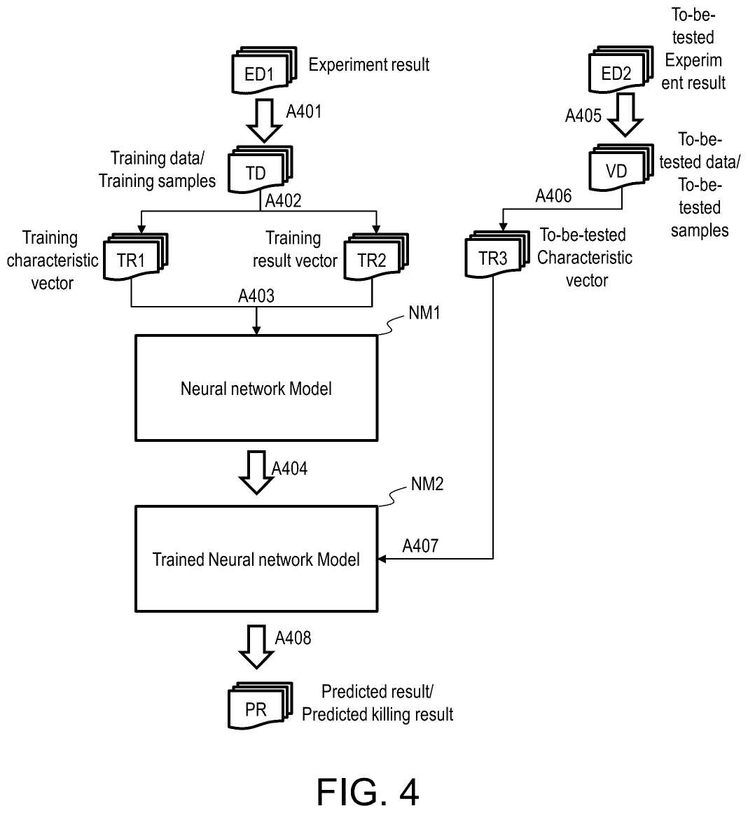

For example, referring to FIG. 4, the processor 110 may generate a plurality of training data TD (A401) according to the experimental result data ED1. The plurality of training data TD can be split into two types of vector data, namely, the training characteristic vector TR1 (also known as training first vector) and the training result vector TR2 (also known as training second vector) (A402). In which, the training characteristic vector TR1 is, for example, an aggregate vector for recording multiple KAR characteristic values of the NK cell; and the training result vector TR2 is, for example, a vector for recording a reduction proportion (%) of the target cancer cell after application of the NK cell.

The training characteristic vector TR1 and the training result vector TR2 are inputted into the neural network model NM1 (A403) for performing supervised learning to obtain the trained neural network model NM2 (A404).

After obtaining the trained neural network model NM2, on the other hand, the processor 110 may generate the to-be-tested data VD (A405) according to the to-be-tested experimental result ED2. The processor 110 recognizes the to-be-tested characteristic vector TR3 (e.g., multiple KAR characteristic values of the to-be-tested NK cell) in the to-be-tested data VD (A406) and inputs the to-be-tested characteristic vector TR3 into the trained neural network model NM2 (A407). Thereafter, the trained neural network model NM2 outputs the prediction result PR (A408) according to the inputted to-be-tested characteristic vector TR3.

In this embodiment, the focus is on evaluating the importance of biomarkers (e.g., KAR characteristic values), which are critical for regression and classification models. Specifically, a neural network approach was utilized for training and prediction. In order to accurately assess the significance of biomarkers, this embodiment utilizes the method proposed by Gevrey et al. in 2003 (“Gevrey, M., Dimopoulos, I., & Lek, S. (2003). Review and comparison of methods to study the contribution of variables in artificial neural network models. Ecological Modelling, 160(3), 249-26”). This method 10 is an analysis of the absolute value of the model weights. In this method, weight can be understood as the strength of connecting each neuron in the neural network, and the absolute value of the weight represents the degree of influence of relevant features on the model prediction results. By analyzing these weights, the biomarkers most important for model predictions can be identified.

It is worth noting that the importance of these biomarkers is consistent across classification models for specific categories. In other words, these markers play an equally important role regardless of the type of forecast. Moreover, this finding helps to understand how the model handles different types of data and also helps to further optimize the performance of the model.

In an embodiment, a backpropagation algorithm is used to train a neural network. This method can effectively adjust the weights in the neural network to improve the prediction accuracy.

More specifically, two different variants of backpropagation are used. One is the elastic backpropagation with weight recurrence proposed by Riedmiller in 1994 (Riedmiller M. (1994) Rprop—Description and Implementation Details. Technical Report. University of Karlsruhe). The other is the version (i) without weighted recursion proposed by Riedmiller and Braun in 1993. Moreover, the modeling architecture provided takes into account a modified version of the convergence of the whole domain proposed by Anastasiadis et al. in 2005 (Anastasiadis A. et. al. (2005) New globally convergent training scheme based on the resilient propagation algorithm. Neurocomputing 64, pages 253-270). The selection of these methods makes the model more efficient and accurate during the learning process.

In addition, the modeling architecture provided in this embodiment also employs a generalized weighting calculation method (Intrator O. and Intrator N. (1993) Using Neural Nets for Interpretation of Nonlinear Models. Proceedings of the Statistical Computing Section, 244-249 San Francisco: American Statistical Society (eds)). This modeling architecture not only considers how to construct the hierarchical structure of a neural network, but also incorporates a series of important parameters, such as learning rate (e.g., set to 0.01), the threshold (e.g., set to 0.01), and the maximum number of steps (e.g., set to 500000), etc.

Referring to FIG. 3, in step S340, the processor 110 determines a quality of the to-be-tested natural killer cell according to the predicted killing result.

In more detail, the step of determining quality of the to-be-tested natural killer cell according to the predicted killing result includes: when a predicted reduction proportion of the target cancer cell is greater than or equal to A, the quality of the to-be-tested natural killer cell is determined to be good; when a predicted reduction proportion of the target cancer cell is less than A and greater than B, the quality of the to-be-tested natural killer cell is determined to be medium; and when a predicted reduction proportion of the target cancer cell is less than or equal to B, the quality of the to-be-tested natural killer cell is determined to be poor, in which A is greater than B.

In one embodiment, A and B are predetermined according to the following variables: the KAR characteristic values and types of target cancer cells, in which the KAR characteristic values comprise: expression ratios of markers of KARs of a natural killer cell. For example, when the target cancer cell is triple-negative breast cancer cells (MDA-MB-231), A is 70% and B is 40%. When the target cancer cell is leukemia cancer cells (K562), A is 50% and B is 36%. The specific values of A and B mentioned above are the comprehensive results defined based on many tests.

In an embodiment, the types and sources of to-be-tested natural killer cells may be plural, and information about each to-be-tested natural killer cell, such as the donor, can be recorded in the cell database 1341. After determining the quality of all of the to-be-tested natural killer cells by the above steps, the processor 110 may further select a portion of the natural killer cells with the highest or higher quality from all of the to-be-tested natural killer cells, and the selected NK cells are used as candidate natural killer cells (also known as clinical natural killer cells) for clinical use. The processor 110 may display relevant information corresponding to one or more clinical natural killer cells on a display of the computing device 100. In another embodiment, the processor 110 may generate a notification message corresponding to the relevant information of one or more clinical natural killer cells and send the notification message to the connected medical control center or related bioengineering department via the communication circuit unit 120. In this way, according to the notification message, healthcare professionals at the Medical Control Center or bioengineered product lines of the relevant bioengineering department could use the selected clinical natural killer cell(s) to perform genetic engineering and expand them to a sufficient number of cells. Then, the NK cell immunotherapy is performed by infusing a sufficient number of the selected clinical natural killer cells into the body of the cancer patients.

In another embodiment, a medical institution may also assess an individual's immune function by drawing blood and analyzing NK cell markers (e.g., KAR characteristic values). In addition, cell manufacturers can analyze NK cell markers to control the function of the NK cell products they produce.

In an embodiment, the processor 110 may retain portions of the generated training data for use in testing the accuracy of the obtained trained neural network model (because the killing result of the retained training data is known).

More specifically, 1000 data sets were randomly generated from the same set of experimental data from donors, and each data set contained a 9:1 ratio distribution. This 9:1 ratio means that from each data set, a large portion of the data (with a ratio of 9) is randomly selected to be used for training the model, while the remaining small portion of the data is used for testing the model. In other words, from the multiple experimental data, 90% of the data is taken for training and 10% of the data is taken for testing in order to generate a data set. Then, from the same experimental data, 90% of the data were randomly taken as training data, and the remaining 10% as test data. By analogy, 1000 data sets are generated.

The methods described above are useful for comprehensively evaluating the performance of a model on different samples. Next, the prediction accuracy was calculated by comparing the difference between the predicted and actual observed values of each model. This comparison will help to understand the effectiveness of the model in practical applications. A high accuracy model means that its predictions are very close to the real data, which is important for subsequent practical applications.

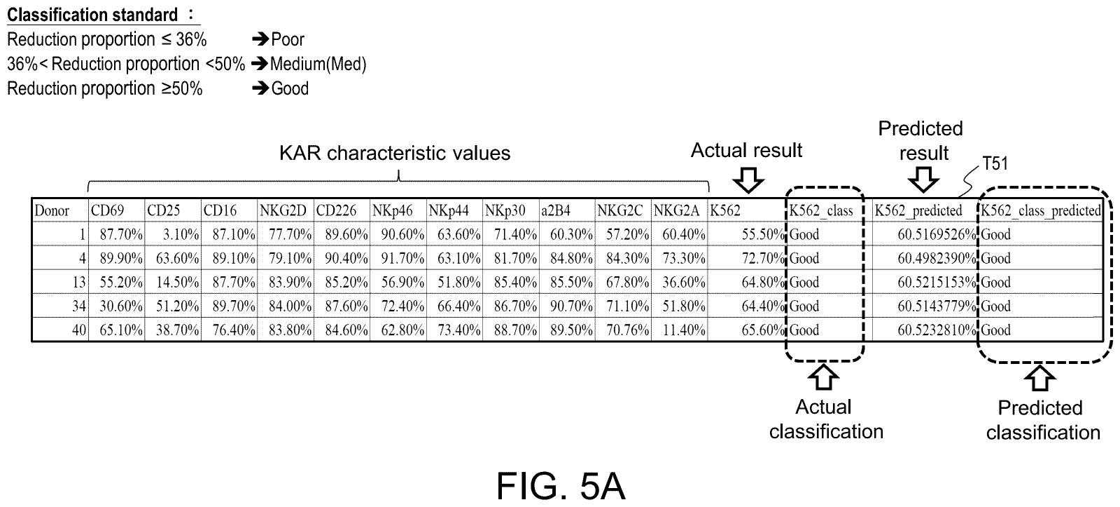

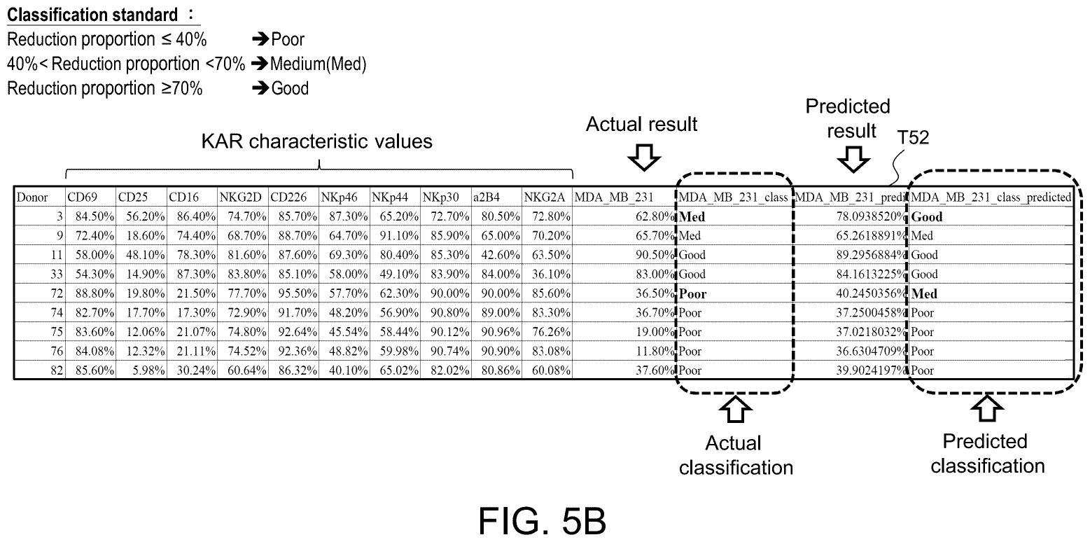

For example, referring to FIG. 5A and Table T51, in which the target cancer cell is K562, the processor 110 retains 5 training data corresponding to the NK cells of 5 donors as test data for validation purposes. Each test data includes 11 KAR characteristic values and the actual killing result. As described above, the processor 110 classifies the NK cells as good (due to a reduction proportion thereof are greater than 50%) according to a classification criterion based on the killing results of the NK cells. The processor 110 also inputs the KAR characteristic values of each of the 5 NK cells into the trained neural network model to obtain the predicted killing results, and then determines that the corresponding predicted classifications are all good.

From the experimental examples described above, the accuracy of the prediction method provided by the present invention is high.

For a further example, referring to FIG. 5B and Table T52, in which the target cancer cell is MDA_MB_231, the processor 110 retains the 9 training data corresponding to the NK cells of the 9 donors as test data for validation purposes. Each test data includes 10 KAR characteristic values and the actual killing result. As described above, the processor 110 classifies the NK cells according to a categorization criterion based on the killing result of the NK cell. In addition, the processor 110 inputs the KAR characteristic values of each of the 9 NK cells into the trained neural network model to obtain the predicted killing results, and then determines the corresponding predicted classification according to the respective killing results. From this validation operation, the accuracy rate is 7/9 or about 78%. For NK cell corresponding to donor #3, the actual quality classification was medium (reduction proportion of 62.8%), but the predicted quality classification was good (reduction proportion of 78.09%). For NK cells corresponding to donor #72, the actual quality classification was poor (reduction proportion of 36.5%), but the predicted quality classification was medium (reduction proportion of 40.24%).

It is worth noting that the methods for predicting NK cell performance provided by the present disclosure can also be used to predict the quality/efficiency of NK cells applied to multiple solid cancers as follows, for example, colon cancer, rectal cancer, lung cancer, breast cancer, liver cancer, head and neck cancer, adenocarcinoma, pancreatic cancer, esophageal cancer, ovarian cancer, and brain cancer, etc., but this disclosure is not limited thereto.

In summary, the present disclosure provides an innovative predictive method and computing device designed for evaluating the killing efficacy of natural killer cells against cancer cells provided by different donors. The method starts by collecting natural killer cell samples from multiple donors and identifying their killing results on the target cancer cells as well as the characteristic factors they possess. These data are used as training data and input into a neural network model. After training, the model can learn and identify the relationship between different characteristic factors and killing effects. After the model has been trained, the characteristic factors of any to-be-tested natural killer cells can be input into the model. The model then outputs a result vector that provides a predicted killing effect, which allows the researchers or physicians to evaluate the potential therapeutic efficacy of the to-be-tested natural killer cells against specific cancer cells. Such prediction not only enhances the potential for individualization of clinical treatments, but also accelerates the screening process for effective treatment options. In addition, after obtaining the predicted quality of each of the to-be-tested multiple natural killer cells, the best or better quality of one or more of the to-be-tested natural killer cells can be selected for the actual application of the natural killer cell immunotherapy program to cancer patients, such that the outcome and quality of life of cancer patients will be greatly improved.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A method for predicting the performance of a Natural Killer (NK) cell, comprising:
generating a plurality of training data according to a characteristic factor and a corresponding killing result against a target cancer cell of each of the natural killer cells;
obtaining a trained neural network model by inputting the plurality of training data into a neural network model;
inputting a to-be-tested input vector corresponding to at least one characteristic factor of a to-be-tested natural killer cell into the trained neural network model to obtain a result vector outputted by the trained neural network model, wherein the result vector indicates a predicted killing result corresponding to the target cancer cell after applying the to-be-tested natural killer cell; and
determining a quality of the to-be-tested natural killer cell according to the predicted killing result.

2. The method for predicting the performance of the NK cell according to claim 1, wherein
the characteristic factor comprises:
a plurality of killer cell activating receptor (KAR) characteristic values, wherein the KAR characteristic values are expression ratios of the KARs,
and wherein the killing result comprises:
a reduction proportion of the target cancer cell.

3. The method for predicting the performance of the NK cell according to claim 2, wherein the step of obtaining the trained neural network model by inputting the plurality of training data into the neural network model comprises:
generating a training first vector corresponding to each natural killer cell according to the characteristic factor of each natural killer cell in the plurality of training data;
generating a training second vector corresponding to the training first vector according to the killing result of each natural killer cell in the plurality of training data; and
inputting the training first vector and the training second vector into the neural network model to obtain the trained neural network model through a supervised learning algorithm.

4. The method for predicting the performance of the NK cell according to claim 3, wherein the supervised learning algorithm comprises one of the following:
a multilayer perceptron (MLP), consisting of a plurality of layers, each layer has a plurality of nodes, wherein the last layer has only one node and an output of the node of the last layer is the result vector; and
a deep learning networks, comprising convolutional neural networks and recurrent neural networks (RNN).

5. The method for predicting the performance of the NK cell according to claim 1, wherein the target cancer cell comprises one of the following types of cancer cells:
a triple-negative breast cancer cell (MDA-MB-231) and a leukemia cancer cell (K562).

6. The method for predicting the performance of the NK cell according to claim 5, wherein when the target cancer cell is the triple-negative breast cancer cell (MDA-MB-231), the plurality of KAR characteristic values comprise characteristic values corresponding to at least one of the following KARs:
NKG2D, CD226, and CD25, wherein the NKG2D, the CD226, and the CD25 are listed based on the associated weights in descending order,
and wherein when the target cancer cell is the leukemia cancer cell (K562), the plurality of KAR characteristic values comprise characteristic values corresponding to at least one of the following KARs:
CD226, NKp46, and CD16, wherein the CD226, the NKp46, and the CD16 are listed based on the associated weights in descending order.

7. The method for predicting the performance of the NK cell according to claim 6,
wherein when the target cancer cell is the triple-negative breast cancer cell (MDA-MB-231), the plurality of KAR characteristic values further comprise characteristic values corresponding to at least one of the following KARs: CD16, CD56, CD69, NKp30, NKp44, and NKp46,
and wherein when the target cancer cell is the leukemia cancer cell (K562), the plurality of KAR characteristic values further comprise characteristic values corresponding to at least one of the following KARs: CD25, CD56, CD69, NKp30, NKp44, and NKG2D.

8. The method for predicting the performance of the NK cell according to claim 7, wherein the plurality of KAR characteristic values further comprise characteristic values corresponding to at least one of the following KARs:
2B4, NKG2A, NKG2C, CD158a/b, CD57, CD62L, CD161, NKp80, and 4-1BB.

9. The method for predicting the performance of the NK cell according to claim 1, wherein the step of determining quality of the to-be-tested natural killer cell according to the predicted killing result comprises:
when a predicted reduction proportion of the target cancer cell is greater than or equal to A, the quality of the to-be-tested natural killer cell is determined to be good;
when a predicted reduction proportion of the target cancer cell is less than A and greater than B, the quality of the to-be-tested natural killer cell is determined to be medium; and
when a predicted reduction proportion of the target cancer cell is less than or equal to B, the quality of the to-be-tested natural killer cell is determined to be poor, wherein A is greater than B.

10. The method for predicting the performance of the NK cell according to claim 9, wherein A and B are predetermined according to the following variables:
the KAR characteristic values and types of target cancer cells,
wherein the KAR characteristic values comprise: expression ratios of markers of KARs of a natural killer cell.

11. The method for predicting the performance of the NK cell according to claim 10, wherein when the target cancer cell is triple-negative breast cancer cells (MDA-MB-231), A is 70% and B is 40%; and
when the target cancer cell is leukemia cancer cells (K562), A is 50% and B is 36%.

12. A computing device, adapted for predicting performance of a natural killer (NK) cell, comprising:
a processor, wherein the processor is configured to:
generate a plurality of training data according to a characteristic factor and a corresponding killing result against a target cancer cell of each of the natural killer cells;
obtain a trained neural network model by inputting the plurality of training data into a neural network model;
input a to-be-tested input vector corresponding to at least one characteristic factor of a to-be-tested natural killer cell into the trained neural network model to obtain a result vector outputted by the trained neural network model, wherein the result vector indicates a predicted killing result corresponding to the target cancer cell after applying the to-be-tested natural killer cell; and
determine a quality of the to-be-tested natural killer cell according to the predicted killing result.
